(19) United States
(12) Patent Application Publication
(10) Pub. No.: US 2025/0176880 A1
(43) Pub. Date: Jun. 5, 2025

(54) METHODS AND SYSTEMS FOR IMPROVING CAPILLARY BLOOD DRAW AND COLLECTION

(71) Applicant: Laboratory Corporation of America Holdings, Burlington, NC (US)

(72) Inventors: Ulrich Schaff, Livermore, CA (US); Jason Ragar, Pittsburg, CA (US); Mitchell Peevler, Pleasanton, CA (US)

(73) Assignee: Laboratory Corporation of America Holdings, Burlington, NC (US)

(21) Appl. No.: 18/968,942

(22) Filed: Dec. 4, 2024

Related U.S. Application Data
(60) Provisional application No. 63/606,512, filed on Dec. 5, 2023.
(60) Provisional application No. 63/653,621, filed on May 30, 2024.

(51) Int. Cl. A61B 5/15

(52) U.S. Cl. CPC A61B 5/150022; A61B 5/150068

(57) ABSTRACT

A capillary blood collection device includes a body with an internal chamber. The body generally includes a first end, a second end, and a port between the first end and the second end and providing access to the internal chamber between the first end and the second end. The first end defines an opening into the internal chamber, and the second end of the body opposite from the first end is compressible and configured to generate a vacuum when compressed. The port may receive at least one of a lancet device or a collection tube.

### REFERENCE TO RELATED APPLICATIONS

This application claims the benefit of and priority to U.S. Provisional Patent Application No. 63/606,512, filed Dec. 5, 2023, and entitled METHODS AND SYSTEMS FOR IMPROVING CAPILLARY BLOOD DRAW AND COLLECTION, and U.S. Provisional Patent Application No. 63/653,621, filed on May 30, 2024, and entitled METHODS AND SYSTEMS FOR VACUUM-ASSISTED CAPILLARY BLOOD DRAW AND COLLECTION, both of which are hereby incorporated by reference in their entireties.

### FIELD OF THE INVENTION

This application relates to systems and methods for obtaining samples of blood and/or other fluids from the body,

### BACKGROUND

Many clinical or diagnostic procedures require a sample of blood or other fluid from a sample. Various techniques may be utilized to obtain such samples, such as but not limited to venous blood collection, arterial blood collection, and capillary blood collection. Capillary blood collection generally includes obtaining capillary blood by puncturing the skin with one quick, continuous, and deliberate stroke of a lancet and collecting the flow of blood with a collection tube. Common sampling sites for capillary blood collection include a person's finger, heel, or ear lobe, with sampling obtained from a person's finger often referred to fingerstick sampling. While capillary blood collection is less invasive compared to other collection techniques, conventional capillary blood collection techniques collect a relatively low volume of the sample over a relatively long collection time. For example, conventional fingerstick sampling techniques obtain a capillary blood sample of 100-600 μL over a collection time of 1-4 minutes. Such limited collection volumes may be insufficient for certain clinical or diagnostic procedures and/or may only be enough for a single attempt of the clinical or diagnostic procedure. Moreover, conventional capillary blood collection techniques are not suitable for self-collection, and assisted collection is required.

### SUMMARY

Embodiments covered by this patent are defined by the claims below, not this summary. This summary is a high-level overview of various embodiments and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, a capillary blood collection device includes an elongated body defining an internal chamber. The elongated body includes a first end, a second end, and a port between the first end and the second end. The port may provide access to the internal chamber between the first end and the second end. In certain embodiments, the first end of the body defines an opening, and the second end of the body opposite from the first end is compressible.

According to various embodiments, a capillary blood collection device includes a body with an internal chamber and a port providing access to the internal chamber. The port may receive at least one of a lancet device or a collection tube. The body is compressible and may generate a vacuum when compressed.

According to some embodiments, a method of collecting a blood sample includes inserting a finger into an internal chamber of a capillary blood collection device, pressing a lancet into a port of the capillary blood collection device and lancing the finger within the internal chamber at a draw site, inserting a collection tube into the port, compressing the capillary blood collection device to initiate a vacuum within the internal chamber, and collecting the blood sample through the port and into the collection tube.

According to various embodiments, a method of collecting at least 1000 μL of a blood sample with a capillary blood collection device inserting a finger into an internal chamber of a capillary blood collection device, pressing a lancet into a port of the capillary blood collection device and lancing the finger within the internal chamber at a draw site, inserting a collection tube into the port, compressing the capillary blood collection device to initiate a vacuum within the internal chamber, and collecting the blood sample through the port and into the collection tube for a collection time of 30-90 seconds.

According to certain embodiments, a capillary blood collection device includes a body with a first end for receiving a human finger into the capillary blood collection device, a compressible second end opposite from the first end, and a port between the first end and the second end for receiving a capillary blood collection device.

According to certain embodiments, a capillary blood collection device includes a body defining an internal chamber. The body also includes a first end, a second end, a port between the first end and the second end and providing access to the internal chamber between the first end and the second end. In various embodiments, the first end defines an opening into the internal chamber and the second end of the body opposite from the first end is compressible. Optionally, the capillary blood collection device includes a flange at the first end, although in other embodiments a flange need not be included,

According to various embodiments, a method of collecting a blood sample includes creating an incision on a lancing site on the skin of a user, applying a capillary blood collection device to the skin of the user such that the lancet site is contained within an opening defined at a first end of the capillary blood collection device, inserting a collection tube into a port of the capillary blood collection device, compressing a second end of the capillary blood collection device to initiate a vacuum within the internal chamber and to cause the capillary blood collection device to adhere to the skin of the user, and collecting the blood sample through the port and into the collection tube.

According to some embodiments, a capillary blood collection device comprising a body comprising a first end for forming a seal against skin of a user, a compressible second end opposite from the first end, and a port between the first end and the second end.

Various implementations described herein can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The specification makes reference to the following appended figures, in which use of like reference numerals in different figures is intended to illustrate like or analogous components.

FIG. 1 illustrates a capillary blood collection system with a capillary blood collection device, a collection tube, and a lancet according to embodiments.

FIG. 2 illustrates the capillary blood collection device and collection tube of the capillary blood collection system of FIG. 1 according to embodiments.

FIG. 3 illustrates the capillary blood collection device of the capillary blood collection system of FIG. 1 according to embodiments.

FIG. 4 illustrates the capillary blood collection device of the capillary blood collection system of FIG. 1 with an attachment device according to embodiments.

FIG. 5 illustrates another capillary blood collection device for the capillary blood collection system of FIG. 1 according to embodiments.

FIG. 6 is a bottom view of the capillary blood collection device of FIG. 5.

FIG. 7 is a side view of the capillary blood collection device of FIG. 5.

FIG. 8 is a sectional view of the capillary blood collection device of FIG. 5.

FIG. 9 illustrates another capillary blood collection device of the capillary blood collection system of FIG. 1 according to embodiments.

FIG. 10 is a sectional view of the capillary blood collection device of FIG. 9.

FIG. 11 illustrates another capillary blood collection device for the capillary blood collection system of FIG. 1 according to embodiments.

FIGS. 12A-D illustrate a process for collecting blood using the capillary blood collection system of FIG. 1 according to embodiments.

FIG. 13 illustrates a capillary blood collection device according to embodiments,

FIG. 14 is a side view cross-section of the capillary blood collection device of FIG. 13.

FIG. 15 is a top view cross-section of the capillary blood collection device of FIG. 13.

FIG. 16 illustrates a capillary blood collection system with another capillary blood collection device, a lance, and a collection tube,

FIG. 17A-D illustrate a process for collecting blood using the capillary blood collection system of FIG. 16 according to embodiments.

FIG. 18 is another view of the capillary blood collection system of FIG. 16 being used to collect a blood sample.

FIG. 19 is another view of the capillary blood collection system of FIG. 16 being used to collect a blood sample.

### DESCRIPTION OF THE INVENTION

Described herein are systems and methods for improving capillary blood draw and collection. In certain embodiments, the systems and methods described herein include a capillary blood collection device for improving capillary blood draw and collection. In certain embodiments, the systems and methods described herein include a capillary blood collection device providing vacuum-assisted capillary blood collection for self-collection and/or assisted collection as desired.

The capillary blood collection device may include a body with an internal chamber and a compressible bulb. An opening at a first end of the body is configured to engage and/or form a seal with skin of a user and/or to receive a finger or digit of a subject into the internal chamber. The body further includes a port between the first end and a second end, for accommodating a collection tube. In various embodiments, the port may accommodate a collecting accessory device (e.g., a lancet device and/or a collection tube).

The body may be made from an elastic and/or compressible material such that the compressible bulb is at least partially compressible, Compression of the compressible bulb may generate a vacuum within the internal chamber. In some cases, the vacuum generated may allow the capillary blood collection device to adhere to the skin of the user (optionally without adhesives such as but not limited to adhesive tape). The vacuum generated may further cause capillary dilation, thereby greatly increasing blood flow. In certain embodiments, the vacuum may be generated after lancing the skin (e.g., using the lancet device), thereby increasing blood flow and blood collection.

In embodiments where the capillary blood device receives the finger, the vacuum on the finger within the internal chamber may cause capillary dilation, thereby greatly increasing blood flow. In certain embodiments, the vacuum may be generated after sticking the finger (e.g., using the lancet device), thereby increasing blood flow and blood collection.

In embodiments where the capillary blood device engages the skin of the user, the capillary blood collection device may be configured to hermetically seal to the collection tube and/or to hermetically seal to the skin of the user.

In certain embodiments, the capillary blood collection devices described herein optionally include one or more features (and sub-combinations thereof) such as but not limited to a monolithic elastomeric structure forming a bulb shape, a first opening or port configured to hermetically seal to a tube, a second opening configured to hermetically seal to skin, a taper from the second hole to the first hole, the second opening may be approximately perpendicular to the first opening. Optionally, a flap or flange may be provided around the base of the second hole to enhance sealing. and/or the lancet and device are not integrated

Compared to traditional capillary blood collection systems, the systems and methods described herein may provide consistent blood draw, and may provide a high volume capillary blood draw.

In various embodiments, the systems and methods described herein may provide consistent capillary blood draw at a high volume in a shorter collection time compared to traditional approaches. As a non-limiting example, where a conventional fingerstick may obtain a collection volume of 100-600 μL over a collection time of 1-4 minutes, the systems and methods described herein may obtain a collection volume of 1000-2050 μL over a collection time of 30-90 seconds. As a non-limiting example, the systems and methods described herein may obtain a collection volume of 0.05 mL to 5.0 mL over a collection time of up to 4 minutes, such as a collection volume of 0.2 mL to 1.0 mL.

In certain embodiments, the system and methods described herein may allow for capillary blood draw from draw locations on a skin of a user that are traditionally not utilized for capillary blood draw. As non-limiting examples, in addition to locations on fingers, heels, or ear lobes, the systems and methods described herein may facilitate capillary blood draw from other locations on the user's body such as but not limited to arms, torso, legs, etc. Compared to traditional approaches, the systems and methods described herein optionally do not require adhesives and may be repositionable on the skin of the user as desired. The systems and methods described herein may allow for multiple draw attempts with one device, and/or may allow for multiple collection tubes to be utilized at a given draw site on the user's skin.

In certain embodiments, the system and methods described herein may provide blood flow directly from a draw location on a user's skin into a collection tube with minimal contact with the body of the collection device. In various embodiments, the vacuum may create a seal between a collection tube in the port and the skin of the user at the opening. In various embodiments, the systems and methods described herein may provide high volume capillary blood draw with equal or lesser pain to the subject compared to traditional fingerstick techniques. In certain embodiments, the system and methods described herein may provide blood flow directly from a finger into a collection tube with minimal contact with the body of the capillary blood collection device. In various embodiments, the vacuum may cause the capillary blood collection device to collapse around the finger within the internal chamber, thereby creating a seal between a collection tube in the port and the finger within the capillary blood collection device. In various embodiments, the systems and methods described herein may provide high volume capillary blood draw without any increase in pain to the subject compared to traditional fingerstick techniques.

Compared to traditional approaches, the systems and methods described herein may be compatible with both self-collection and assisted collection of the blood sample. In certain embodiments, the systems and methods described herein may be compatible with existing lancet devices and/or collection tubes, thereby providing an easy retrofit system for obtaining high volume capillary blood draw. The capillary blood collection devices described herein may be low cost and provide material savings while also providing a streamlined process for obtaining high volume capillary blood draw.

Various other benefits and advantages may be realized with the systems and methods described herein, and the aforementioned benefits and advantages should not be considered limiting.

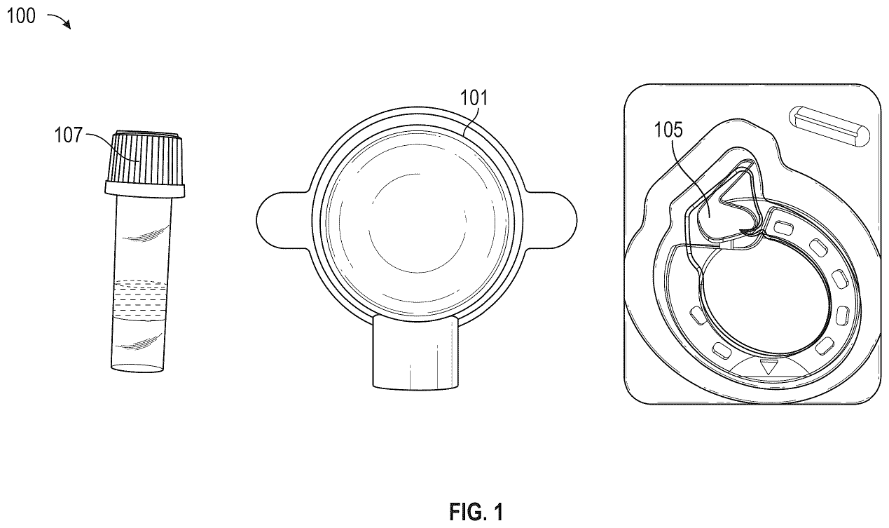

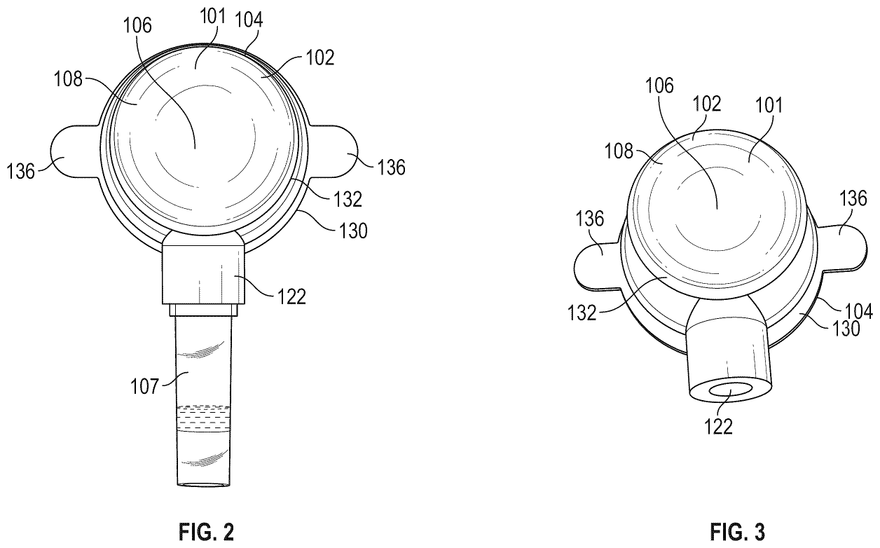

FIGS. 1-4 illustrates a capillary blood draw system 100 according to embodiments. The capillary blood draw system 100 generally includes at least a capillary blood collection device 101, and optionally includes one or more of a collection tube 107 and/or a lancet device 105.

The capillary blood collection device 101 includes generally includes body 102 with first end 104, second end 106 opposite from the first end 104, and an internal chamber 110. In certain embodiments, and as discussed in detail below, the first end 104 of the capillary blood collection device 101 may be positioned against the skin of a user and the collection tube 107 may be received within a port 122. In various embodiments, the body 102 may hermetically seal to the skin of the user and may hermetically seal to the collection tube 107.

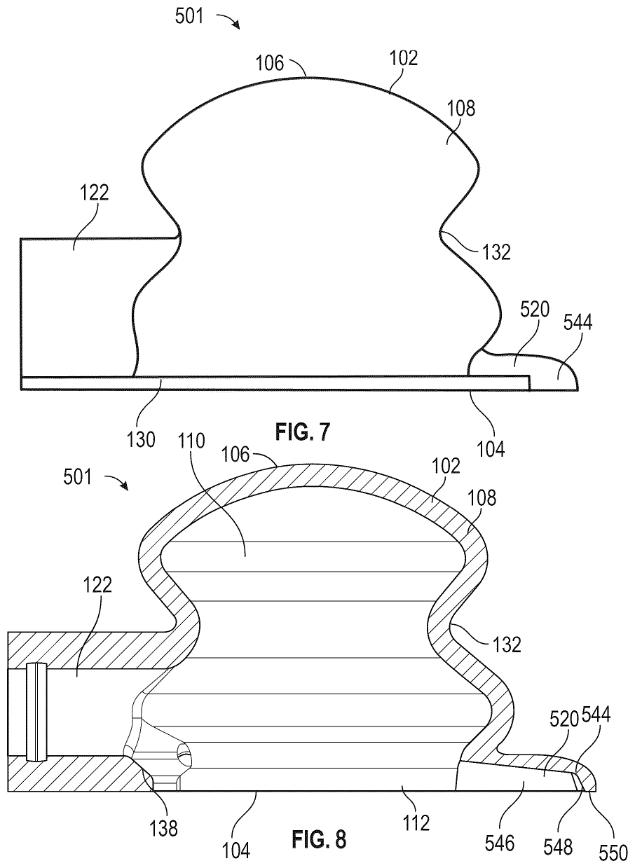

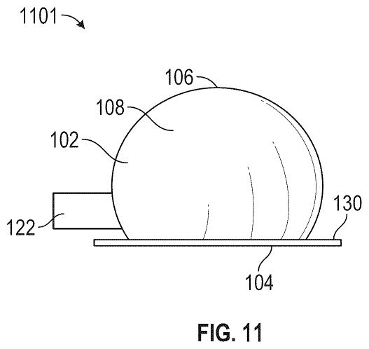

The body 102 may have various shapes or profiles as desired. In certain embodiments, the body 102 includes a bulb region 108 which includes the second end 106. The bulb region 108 may have various shapes or profiles as desired. The bulb region 108 may be configured to be compressed by manual pressing toward the skin of the user. As non-limiting examples, FIGS. 1-10 illustrate the bulb region 108 with a double dome profile and/or comprises two or more bellows/lobes while FIG. 11 illustrates a bulb region 108 with a single dome or lobe. In other non-limiting examples, the bulb region 108 may have other shapes such as but not limited to rectangular, ellipsoid, hemispherical, combinations thereof, and/or as otherwise desired.

In certain embodiments, the body 102 is constructed from an elastomeric and/or compressible material such that at least a portion of the body 102 is compressible, Various materials may be utilized as desired, such as but not limited to elastomeric materials, silicone, thermoplastic elastomer, polyurethane, various types of rubbers, and/or other suitable materials or combinations of materials as desired. In some non-limiting examples, the material of the body 102 may have a hardness of 10-80 Shore A durometer, such as 10 Shore A durometer, 20 Shore A durometer, 30 Shore A durometer, 40 Shore A durometer, 50 Shore A durometer, 60 Shore A durometer, 70 Shore A durometer, and/or 80 Shore A durometer, In various embodiments, the material of the body 102 at least at the first end 104 may conform to a surface of skin of the user. In some embodiments, the material of the body 102 may allow for adhering to the skin of the user via vacuum without adhesive, tape, and/or other adhering mechanisms or devices. In certain embodiments, the body 102 may be a monolithic structure formed from various suitable forming techniques. In one non-limiting example, the body 102 may be a monolithic elastomeric structure formed via a 1-piece molding technique. In certain embodiments, at least the bulb region 108 includes the elastomeric material such that the bulb region 108 may be selectively compressed. As discussed in detail below, in use, compression of at least the bulb region 108 may generate a vacuum within the internal chamber 110.

A wall thickness of the body 102 may be various thicknesses as desired, As non-limiting examples, the wall thickness of the body 102 may be from about 1.0 mm to about 8.0 mm, such as about 1.0 mm, about 1,5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, and/or about 0.8 mm. In one non-limiting example, the wall thickness of the body 102 may be from about 1.5 mm to about 4.0 mm. In other embodiments, the body 102 may have other wall thicknesses as desired.

In various embodiments, an opening 112 (see, e.g., FIGS. 6, 8, and 10) is defined in the first end 104 and provides access to the internal chamber 110. The opening 112 may have various shapes or profiles as desired. Thus, while the opening 112 is illustrated with a generally circular shape, in other embodiments, it need not be and instead may be other shapes such as rectangular, ellipsoid, polygonal, and/or as otherwise desired. In some non-limiting examples where the capillary blood collection device 101 is configured to engage the skin of the user, a maximum transverse dimension (e.g., a diameter for a circular shape) of the opening 112 may be from about 10 mm to about 50 mm, such as about 10 mm, about 15 mm, about 20 mm, about 25 mm, about 30 mm, about 35 mm, about 40 mm, about 45 mm, and/or about 50 mm. In one non-limiting example, the maximum transverse dimension of the opening 112 may be from about 15 mm to about 40 mm. In other embodiments, the maximum transverse dimension may be other dimensions as desired. In certain embodiments, the opening 112 may have a maximum transverse dimension sufficient to encircle or surround a draw location (e.g., as formed by the lancet device 105) on the skin of the user.

Optionally, the first end 104 of the body 102 includes a flange 130. As such, while the flange 130 is illustrated, in other embodiments, the capillary blood collection device 101 may omit the flange 130. In embodiments without the flange 130, the surface of the body 102 at the first end 104 may engage the skin of the user. When the flange 130 is included, the flange 130 may serve to stabilize the opening 112, provide a larger sealing surface, and/or improve success rate. In various embodiments, the flange 130 may facilitate contact between the capillary blood collection device 101 and the skin of the user, sealing of the internal chamber 110, and/or may facilitate the generation of the vacuum within the internal chamber 110.

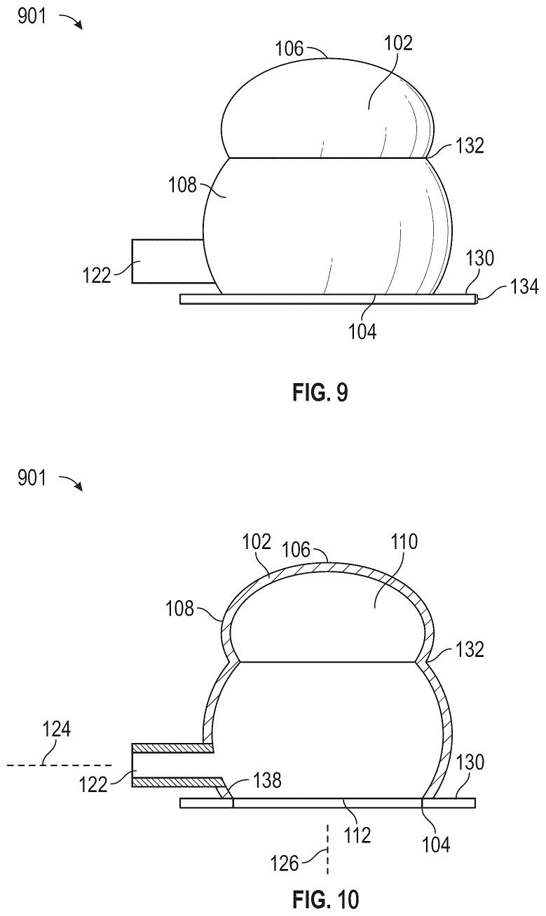

The flange 130 may have various shapes or profiles as desired. In certain embodiments, the flange 130 has a thickness 134 (see, e.g., FIG. 9) promoting contact between the first end 104 and the skin of the user. As non-limiting examples, the thickness 134 may be from about 1.0 mm to about 10.0 mm, such as about 1.0 mm, about 1.5 mm, about 2.0 mm, about 2.5 mm, about 3.0 mm, about 3.5 mm, about 4,0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, and/or about 10.0 mm. In other embodiments, the flange 130 may have other thicknesses as desired.

In certain embodiments, the flange 130 optionally includes one or more removal tabs 136 extending outwards from the flange 130. The removal tabs 136 may facilitate removal of the capillary blood collection device 101 from the skin of the user and/or breaking the vacuum within the capillary blood collection device 101. The number, shape, and location of the removal tabs 136 should not be considered limiting.

In certain embodiments, the bulb region 108 defines one or more grooves 132, When the grooves 132 are included, the grooves 132 may extend partially or completely around the perimeter of the bulb region 108. In some embodiments, the grooves 132 may facilitate and/or promote compression of the bulb region 108 (e.g., to generate the vacuum) and/or may facilitate handling by a user. The number and location of grooves 132 illustrated should not be considered limiting. In other embodiments, the bulb region 108 need not include the grooves 132.

As mentioned, in certain embodiments, at least the bulb region 108 includes the elastomeric material such that the bulb region 108 may be selectively compressed. As discussed in detail below, in use, compression of at least the bulb region 108 may generate a vacuum within the internal chamber 110. In certain embodiments, compression of the bulb region 108 may generate a pressure from about −5 to about −80 kPa. In some non-limiting examples, the pressure generated may be from about −15 kPa to about −50 kPa.

The port 122 of the body 102 extends from the body 102 at a location between the first end 104 and the second end 106. In some embodiments, the port 122 at the intermediate location between the ends 104, 106 may allow the port 122 to accommodate the collection tube 107 offset from the skin of the user. In certain embodiments, and as best illustrated in FIG. 10, an axis 124 of the port 122 extends at an angle relative to an axis 126 of the opening 112. In some embodiments, the axis 124 of the port is substantially perpendicular to the axis 126 of the opening 112. However, in other embodiments, the axis 124 need not be perpendicular to the axis 126. In various embodiments, and as best illustrated in FIGS. 8 and 10, a transverse dimension of the port 122 is less than the transverse dimension of the opening 112.

In various embodiments, and as best illustrated in FIGS. 8 and 10, a guiding surface 138 may extend from the opening 112 to the port 122 within the internal chamber 110. In certain embodiments, the guiding surface 138 may be sloped, tapered, and/or otherwise shaped and/or placed to facilitate a flow of blood drawn through the opening 112 into the port 122.

Optionally, the capillary blood collection device 101 includes one or more venting features for selectively venting air from within the chamber 110. In other embodiments, venting features need not be included. When the venting features are included, such venting features may be provided at or on various portions of the body 102 as desired, such as on the bulb region 108, at the second end 106, at the first end 104, and/or at the flange 130. The number, type, and location of the one or more venting features should not be considered limiting. In certain embodiments, the one or more venting features are configured to allow venting of the internal chamber 110 upon compression of the bulb region 108. In some embodiments, the one or more venting features are self-sealing venting features. Non-limiting examples of venting features include, but are not limited to, duckbill valves, umbrella valves, or other suitable features or combinations of features as desired. As non-limiting examples, the bulb region 108 may include a venting feature which includes a valve which is a slit in the body 102. Additionally, or alternatively, the first end 104 and/or the flange 130 may include a valve as a venting feature and which includes a slit in the body 102. Additionally, or alternatively, as another non-limiting example, an integrated valve or venting feature may include a valve opening wherein a transverse dimension of the valve opening is less than a transverse dimension of the opening in the first end. In such embodiments. Optionally, the valve opening may have tapered sides and/or may close under negative pressure by drawing up the skin to plug the opening. Other venting features may be utilized as desired.

The lancet device 105 may be various devices and/or mechanisms suitable for puncturing the skin of a user for capillary blood collection may be utilized as desired. As a non-limiting example, the lancet device 405 may be a lancet device sold under the trade name Unistik™ by Owen Mumford™ or may be an incision device sold under the trade name Gentleheel™ by GRI; however, in other embodiments, other lancet devices 405 may be utilized. In some embodiments, the lancet device 105 may be various suitable devices for forming a cut depth between 0.5 mm and 2.5 mm, such as about 0.5 mm, about 1.0 mm, about 1.5 mm, about 2.0 mm, and/or about 2.5 mm. In one non-limiting examples, the lancet device 105 may have a cut depth of about 2.0 mm, In various embodiments, the lancet device 105 may be various suitable devices for forming a cut width between 0.5 mm and 5.0 mm, such as about 0.5 mm, about 1.0 mm, about 1.5 mm, about 2.0 mm, about 2.5 mm, about 3.0 mm, about 3.5 mm, about 4.0 mm, about 4.5 mm, and/or about 5.0 mm. In one non-limiting example, the cut width may be about 3.0 mm. In other embodiments, the cut depth and/or the cut width may be other dimensions as desired, In some embodiments, the lancet device 105 may actuate via various mechanisms or motions as desired, such as but not limited to a jabbing motion or a sweeping motion.

The collection tube 107 may be various suitable collection tubes as desired. As a non-limiting example, the collection tube 107 may be a collection device sold under the trade name BD Microtainer® by Becton Dickinson; however, in other embodiments, other collection tubes 407 may be utilized as desired. In various embodiments, and as discussed in detail below, the lancet device 105 and the collection tube 107 may be positioned within the port 122 during a sampling process.

In certain embodiments, and as illustrated in FIG. 1, the components of the capillary blood draw system 100 are separate and/or not integrated, thereby improving modularity of the capillary blood draw system 100. As examples, the particular collection tube 107 and/or the lancet device 105 illustrated in FIG. 1 should not be considered limiting, and in various embodiments, the capillary blood collection device 101 may be compatible with a plurality of types of collection tubes 107 and/or a plurality of types of lancet devices 105. The modular capillary blood draw system 100 may further allow for a plurality of collection tubes 107 to be utilized with a single capillary blood collection device 101 at a single draw location on the skin of the user. In certain embodiments, the modular capillary blood draw system 100 may further allow for positioning of components relative to each other and/or relative to the skin of the user during a blood collection process.

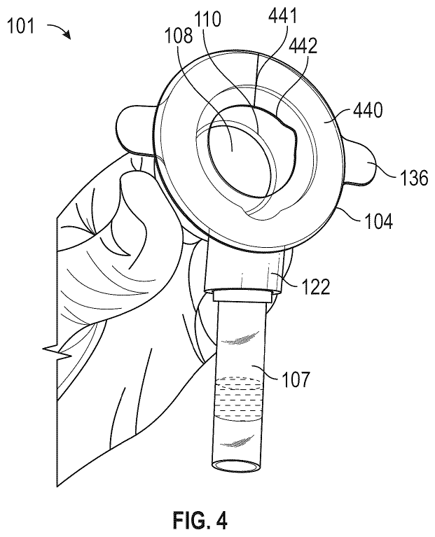

Referring to FIG. 4, the capillary blood collection device 101 optionally includes an attachment device 440 at the first end 104. In such embodiments, the attachment device 440 may promote attachment of the capillary blood collection device 101 to a skin surface that is textured and/or otherwise not smooth (e.g., due to bair, surface features, etc.), In some embodiments, the attachment device 440 may be constructed from a material that is softer than that of the body 102. For example, the attachment device 440 may be constructed from silicone with hardness of 10-20 Shore A durometer. In one non-limiting example, the attachment device 440 may be a gasket, although in other embodiments, other attachment devices 440 may be utilized. Optionally, the attachment device 440 may include a venting feature to facilitate air escape during actuation. As a non-limiting example, the venting feature in the attachment device 440 may include a shallow slit feature and/or channel 441. In other embodiments, other venting features may be utilized as desired.

As illustrated in FIG. 4, the attachment device 440 defines an attachment device opening 442. In some embodiments, a maximum transverse dimension of the attachment device opening 442 is less than the transverse dimension of the opening 112. In such embodiments, the smaller attachment device opening 442 may further promote and/or facilitate adhering of the capillary blood collection device 101 to the skin of the user. In some embodiments, a thickness of the attachment device 440 may be greater than the thickness 134 of the flange 130. In non-limiting examples, the thickness of the attachment device 440 may be from about 0.1 mm to about 1.0 mm, such as about 0.1 mm, about 0.2 mm, about 0.3 mm, about 0.4 mm, about 0.5 mm, about 0.6 mm, about 0.7 mm, about 0,8 mm, about 0.9 mm, and/or about 1.0 mm. In other embodiments, the attachment device 440 may have other thicknesses as desired.

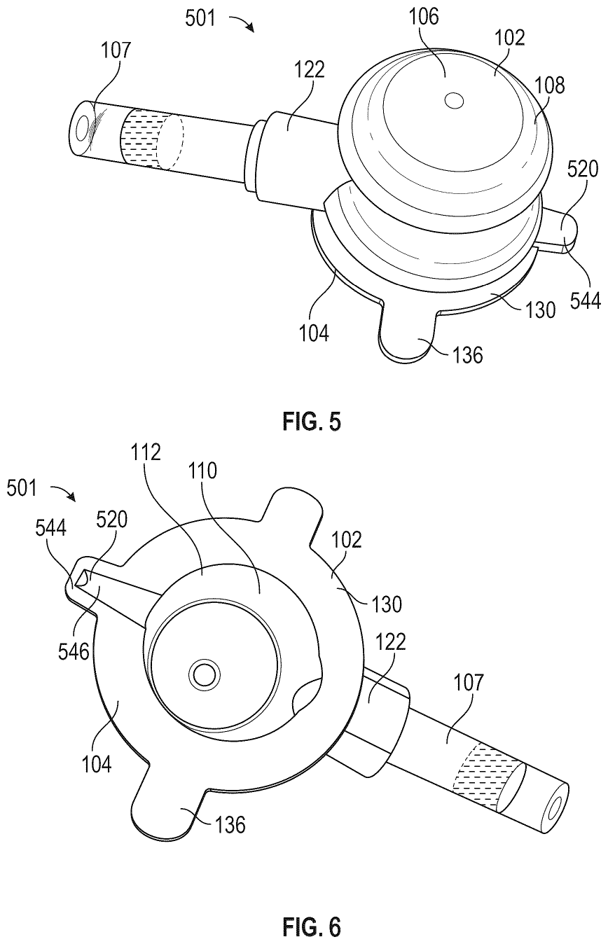

FIGS. 5-8 illustrate another capillary blood collection device 501 that is substantially similar to the capillary blood collection device 101. Compared to the capillary blood collection device 101, the capillary blood collection device 501 includes a venting feature 520.

In the embodiment illustrated, the venting feature 520 optionally extends outwards from the body 102 and includes a flap 544 with a channel 546. In some embodiments, the channel 546 may be provided on a skin-facing side of the flap 544 and may connect or extend to the internal chamber 110. Optionally, the location of the venting feature 520 is opposite from the port 122, although it need not be in other embodiments. The venting feature 520 may be configured such that a compression of the capillary blood collection device 501 causes the flap 544 to lift off of the skin, causing air to be expelled from the internal chamber 110 preferentially through the venting feature 520 to the exterior of the device, creating a vacuum when the compression is released. Optionally, an edge 548 of the channel 546 is separated from the exterior of the capillary blood collection device 501 by a flange 550. The flange 550 may have various shapes, dimensions, and/or profiles as desired. In some non-limiting examples, the flange 550 may have a length from about 0.5 mm to about 4 mm. In other examples, flanges of other dimensions may be utilized as desired. The channel 546 may have various shapes, dimensions, and/or profiles as desired, As non-limiting examples, the channel 546 may have a depth from about 0.5 mm to about 5 mm, a width from about 1 mm to about 10 mm, and/or a length of from about 2 mm to about 20 mm. Other dimensions may be utilized as desired. Moreover, as illustrated, one or more dimensions optionally may be tapered and/or have a non-uniform dimension. Other venting features 520 may be utilized as desired, and the particular venting feature 520 illustrated should not be considered limiting.

FIGS. 9 and 10 illustrate a capillary blood collection device 901 that is substantially similar to the capillary blood collection device 101 except that the domes or lobes of the body 102 have a different profile or shape compared to that of the capillary blood collection device 101

FIG. 11 illustrates a capillary blood collection device 1101 that is substantially similar to the capillary blood collection device 101 except that the body 102 has a hemisphere or single dome shape compared to that of the capillary blood collection device 101.

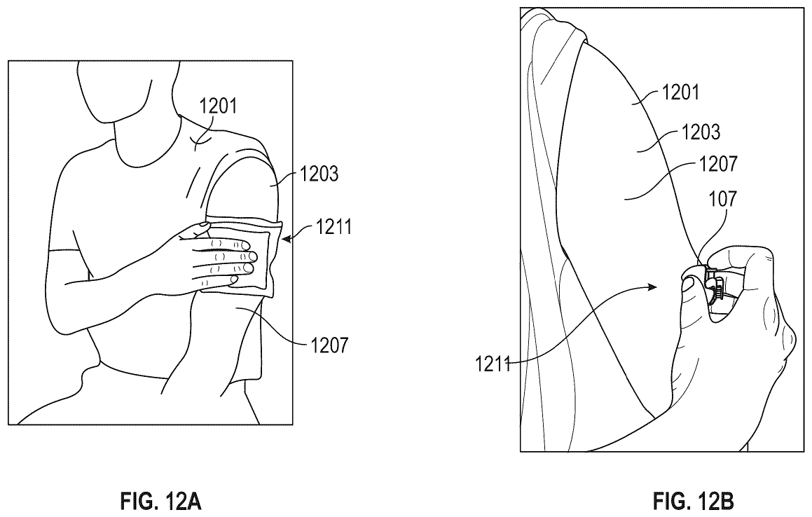

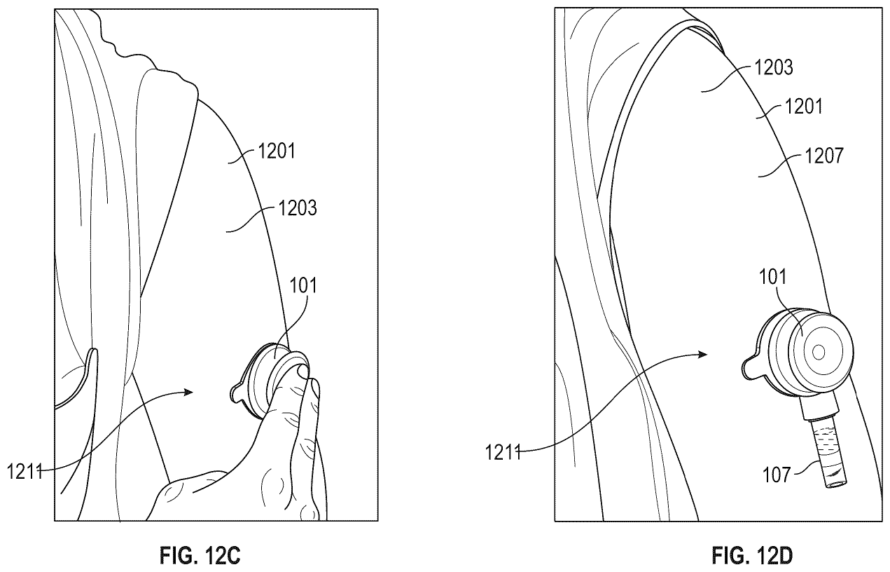

Referring to FIGS. 12A-D, a method of collecting capillary blood using the capillary blood collection devices described herein is illustrated in greater detail. While blood collection device 101 is illustrated, the method is equally applicable to the other blood collection devices.

Optionally, and with reference to FIG. 12A, the method includes wiping, cleaning, and/or otherwise preparing a draw site 1211 before using the capillary blood collection device 101. In some embodiments, and as illustrated in FIG. 12A, the method optionally includes warming the skin 1203 of the user 1201 at the desired draw site 1211. In some embodiments, the draw site 1211 may be on an arm 1207 of the user 1201, although in other embodiments it may be at other locations such as but not limited to an upper arm, forearm, palm of hand, leg, heel, etc. The skin 1203 may be warmed to various temperatures as desired. In one non-limiting example, the skin 1203 may be warmed to a temperature of about 37° C. to about 42° C.

As illustrated in FIG. 12B, regardless of whether the skin 1203 is warmed, the method includes lancing or cutting the skin 1203 at the draw site 1211 with the lancet device 105.

As illustrated in FIG. 12C, the method includes applying the capillary blood collection device 101 to the draw site 1211 with the draw site 1211 contained within the opening 112. The method optionally includes attaching the collection tube 107 at the port 122 of the capillary blood collection device 101 at any time prior to applying the capillary blood collection device 101 to the skin, although the collection tube 107 may be attached after applying the capillary blood collection device 101 to the skin as desired.

As illustrated in FIG. 12C, the method includes compressing the capillary blood collection device 101 to expel air, and the capillary blood collection device 101 is allowed to decompress while pressed against the skin. In various embodiments, compression the capillary blood collection device 101 includes compressing (e.g., squeezing) the bulb region 108 and/or the second end 106. Compressing the bulb region 108 and/or the second end 106 may cause air to be vented from the internal chamber 110, thereby initiating vacuum within the internal chamber 110 when the compression is released. In certain embodiments, compressing the bulb region 108 and/or the second end 106 to initiate the vacuum may cause the body 102 to collapse against the skin 1203 and create a seal between the skin 1203 and the capillary blood collection device 101. Air may escape from the interface between the skin and the device and/or via venting features in the body 102 as desired, Additionally, or alternatively, compressing the bulb region 108 may be done prior to applying the device to the skin to bypass the issue of venting air. As a non-limiting example, the bulb region 108 could be compressed by squeezing prior to application to the skin, and released after application to the skin 1203. In such embodiments, the capillary blood collection device 101 optionally need not include any venting feature.

As illustrated in FIG. 12C, in certain embodiments, the collection tube 107 is oriented vertically downward.

The vacuum initiated by the capillary blood collection device 101 causes capillary dilation and increases blood flow from the draw site 1211 into the collection tube 107. Larger diameter (i.e., dilated) capillaries and microvasculature increase local blood supply and reduce flow resistance after lancing, thereby providing an increased blood flow to the collection tube 107. In certain embodiments, a single compression/initiation of the vacuum may be utilized. However, in other embodiments such as where a sample is needed for multiple collection tubes 107, the capillary blood collection device 101 may be further compressed to initiate successive vacuums, thereby providing the ability for a multi-draw capacity from the same draw site 1211 due to the increased blood flow.

In certain embodiments, a partial vacuum is generated by the rebound of the capillary blood collection device 101, and the partial vacuum causes the capillary blood collection device 101 to adhere to the skin 1203 as illustrated in FIG. 12D.

In certain embodiments, the method includes maintaining the capillary blood collection device 101 on the skin 1203 for a duration to collect a desired volume of blood. In one non-limiting example, the method includes maintaining the capillary blood collection device 101 on the skin 803 for a duration of up to about 4 minutes to collect 0.1 to 1.0 mL of blood.

In various embodiments, after the duration and/or after a desired volume of blood is collected, the method includes removing the capillary blood collection device 101 from the skin 1203. In some embodiments, the method includes peeling and/or removing the capillary blood collection device 101 from the skin 1203, optionally using the flange 130 and/or a removal tab 136. In various embodiments, the method optionally includes removing the collection tube 107 from the port 122 before removing the capillary blood collection device 101 from the skin 1203 or after removing the capillary blood collection device 101 from the skin 1203.

Optionally, the method includes inserting a second collection tube 107 into the port 122 and re-actuating the capillary blood collection device 101 to further draw blood from the same draw site 1211. Such steps may be repeated as many times and with as many collection tubes 107 as desired.

Optionally, the method includes making lancing or cutting the skin 1203 at a second draw site different from the draw site 1211, optionally using a second lancet device 105 (although the same lancet device 105 may be utilized). Optionally, a second blood collection may be attempted using the same capillary blood collection device 101 at the second draw site.

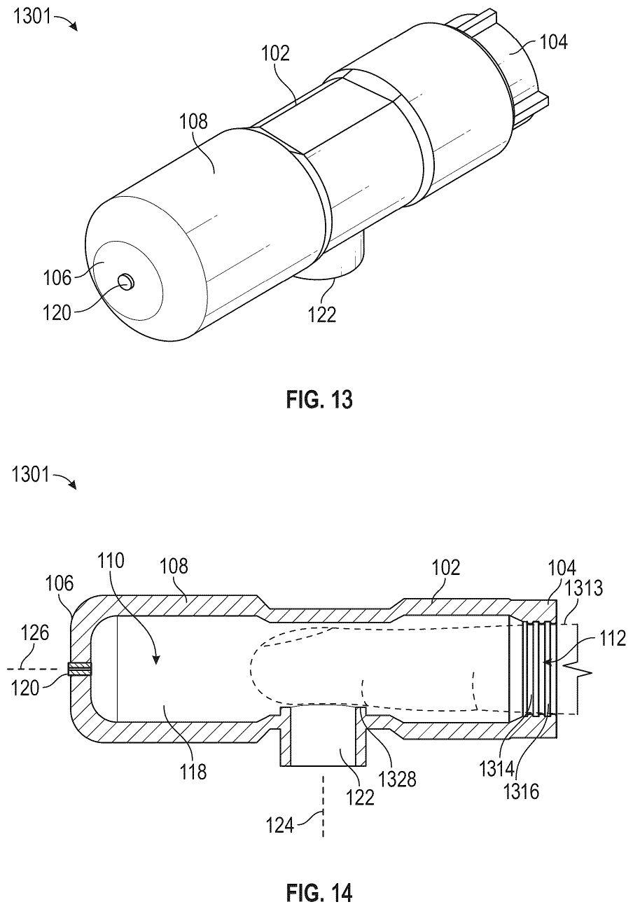

FIGS. 13-15 illustrate another capillary blood collection device 1301 according to embodiments and that is similar to the capillary blood collection device 101. Compared to the capillary blood collection device 101, the body 102 of the capillary blood collection device 1301 generally is elongated and having the first end 104, the second end 106 opposite from the first end 104, and the internal chamber 110.

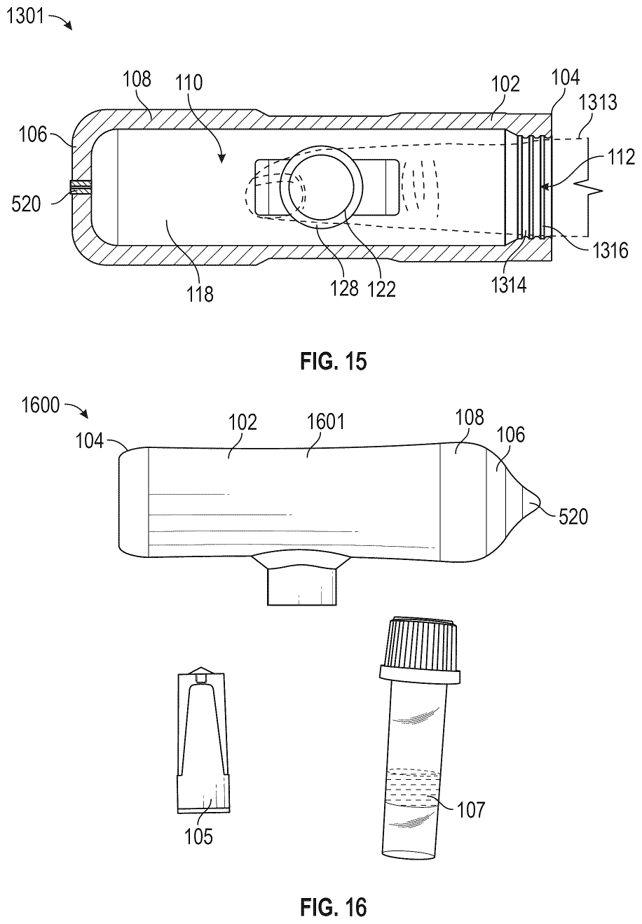

Similar to the capillary blood collection device 101, as best illustrated in FIGS. 14 and 15, the first end 104 of the body 102 of the capillary blood collection device 1301 may define the opening 112 providing access to the internal chamber 110. However, compared to the capillary blood collection device 101, the opening 112 of the capillary blood collection device 1301 may receive a finger (represented by dashed lines 1313 in FIGS. 14 and 15) of a subject during use.

In certain embodiments, and as illustrated in FIGS. 14 and 15, the first end 104 of the body 102 of the capillary blood collection device 1301 optionally includes one or more sealing features 1314 for forming a seal with the finger 1313. In the embodiment illustrated, the one or more sealing features 1314 includes a plurality of sealing ribs 1316 on an inner surface 118 of the internal chamber 110. However, the number, shape, location, and type of sealing feature 1314 should not be considered limiting. As examples, in other embodiments, the one or more sealing features 1314 may be a single sealing rib 1316 and/or one or more sealing features other than sealing ribs 1316 (e.g., sealing lips, rings, gaskets, liners, etc.). In some embodiments, the one of more sealing features 1314 optionally may have a dimension and/or other feature such that the one or more sealing features 1314 at least partially constrict the finger 1313. As discussed in detail below, constriction of the finger 1313 by the one or more sealing features 1314 may increase dilation during a collection process.

Optionally, and similar to the capillary blood collection device 501, the capillary blood collection device 1301 may include one or more venting features 520 for selectively venting air from within the internal chamber 110. Optionally, and compared to the capillary blood collection device 501 where the venting features 520 are at the first end 104, the second end 106 and/or the bulb region 108 of the body 102 of the capillary blood collection device 1301 may include the one or more venting features 1320. However, as previously mentioned, the number, type, and location of the one or more venting features 520 should not be considered limiting. In certain embodiments, the one or more venting features 520 are configured to allow venting of the internal chamber 110 upon compression of the bulb region 108. In some embodiments, the one or more venting features 520 are self-sealing venting features, Non-limiting examples of venting features 520 include, but are not limited to, duckbill valves, umbrella valves, or other suitable features or combinations of features as desired.

As best illustrated in FIGS. 14 and 15, between the first end 104 and the second end 106, the body 102 of the capillary blood collection device 1301 includes the port 122 providing access to the internal chamber 110. In some embodiments, a diameter (or transverse dimension) of the port 122 is less than a diameter (or transverse dimension) of the opening 112 and/or the internal chamber 110, although it need not in other embodiments. As best illustrated in FIG. 14, the port 122 includes the center axis 124 which intersects the center axis 126 of the internal chamber 110/opening 112. In the embodiment illustrated, the center axis 124 is substantially perpendicular to the center axis 126. However, in other embodiments, the center axis 124 may be at various angles relative to the center axis 126 as desired and need not be perpendicular.

In some embodiments, and as illustrated in FIGS. 14 and 15, the body 102 of the capillary blood collection device 1301 optionally includes a finger seal 1328 within the internal chamber 110. The finger seal 1328 may extend inwards from the inner surface 118 (e.g., towards the center axis 126) and may be aligned with the port 122. In certain embodiments, the finger seal 1328 may contact the finger 1313 of the subject during use, which may minimize contact between a draw area and a material of the body 102. In various embodiments, and as discussed in detail below, the finger seal 1328 may facilitate the creations of a seal between a collection tube and the finger 1313, thereby allowing blood flow direction from the finger 1313 into the collection tube 107.

FIGS. 16 illustrates another capillary blood collection system 1600 with a capillary blood collection device 1601, the lancet device 105, and the collection tube 107. The capillary blood collection device 1601 is substantially similar to the capillary blood collection device 1301 except that a shape of the body 102 of the capillary blood collection device 400 is different from a shape of the body 102 of the capillary blood collection device 1301. The lancet device 105 may be various suitable lancet devices as desired. As a non-limiting example, the lancet device 105 may be a lancet device sold under the trade name Unistik™ by Owen Mumford™ or may be an incision device sold under the trade name Gentleheel™ by GRI; however, in other embodiments, other lancet devices 105 may be utilized. Similarly, the collection tube 107 may be various suitable collection tubes as desired. As a non-limiting example, the collection tube 107 may be a collection device sold under the trade name BD Microtainer® by Becton Dickinson; however, in other embodiments, other collection tubes 407 may be utilized as desired. In various embodiments, and as discussed in detail below, the lancet device 405 and the collection tube 107 may be positioned within the port 122 during a sampling process.

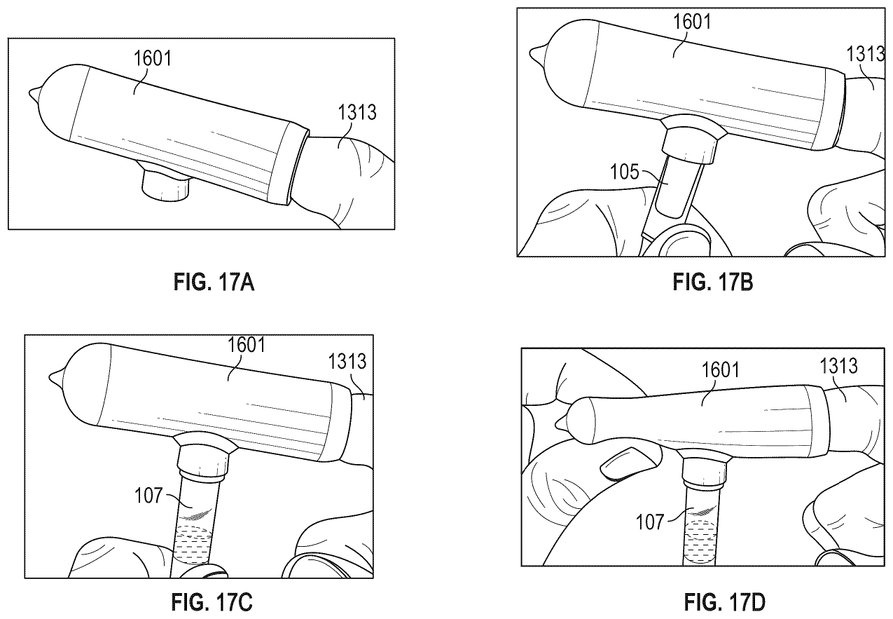

A method of collecting a capillary blood sample will be described in greater detail with reference to FIGS. 17A-D, 18, and 19, While the method is described with the capillary blood collection device 1601, the method may be utilized with any capillary blood collection device consistent with the embodiments described herein, including but not limited to the capillary blood collection device 1301.

Referring to FIG. 17A, the method may include inserting the finger 1313 of the subject through the opening 112 and into the internal chamber 110 of the capillary blood collection device 1601. Optionally, the method includes wiping, cleaning, and/or otherwise preparing the finger 1313 before inserting the finger 1313 into the capillary blood collection device 1601.

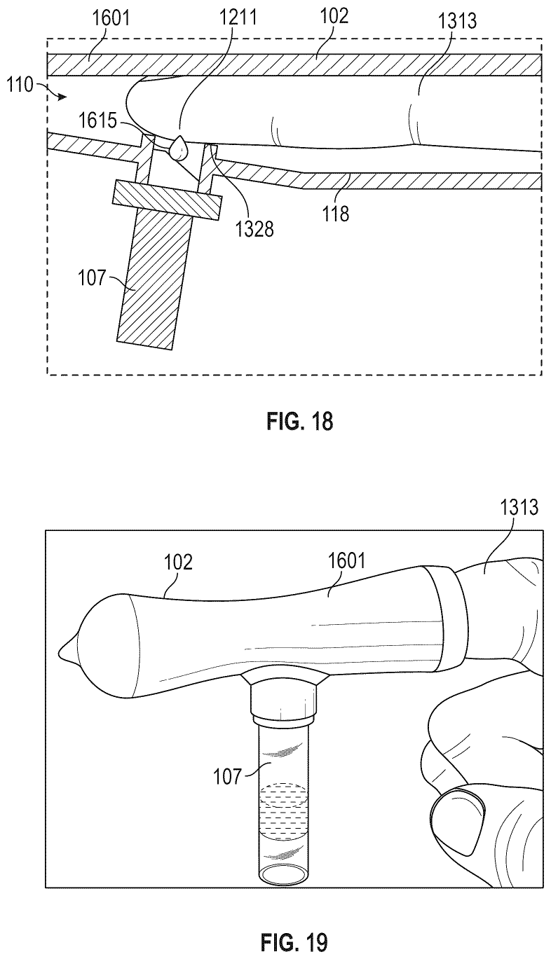

Referring to FIG. 17B, after the finger 1313 is inserted into the capillary blood collection device 1601, the method includes pressing and/or otherwise positioning the lancet device 105 within the port 122. The method may include causing the lancet device 105 to lance the finger 1313 at the draw site 1211 (see FIG. 18) on the finger 1313 and within the internal chamber 110.

In one non-limiting example, causing the lancet device 105 to lance the finger 1313 optionally may include pressing the lancet device 105 into the port 122 until audible and/or tactile feedback is received (e.g., an audible “click”), thereby indicating that the lancing operation is completed.

Referring to FIGS. 17B-D, 18, and 19, after the finger 1313 has been lanced by the lancet device 105, the method includes removing the lancet device 105 from the port 122 and inserting the collection tube 107 into the port 122. Referring to FIG. 17D, after the collection tube 107 is positioned within the port 122, the method includes compressing (e.g., squeezing) the bulb region 108 and/or the second end 106. Compressing the bulb region 108 and/or the second end 106 may cause air to be vented from the internal chamber 110 (e.g., via the venting feature 520), thereby initiating vacuum within the internal chamber 110.

Referring to FIGS. 17D, 18, and 19, in certain embodiments, compressing the bulb region 108 and/or the second end 106 to initiate the vacuum may cause the body 102 to collapse around the finger 1313 and create a seal between the capillary blood collection device 1601 and the finger 1313. As non-limiting examples, initiating the vacuum may cause the finger seal 1328 to contact an area of the finger 1313 adjacent to the draw site 1211 and create a seal between the collection tube 107 and the finger 1313.

As illustrated in FIG. 18, the vacuum initiated by the capillary blood collection device 1601 causes capillary dilation and increases blood flow 1615 from the finger 1313 into the collection tube 107. In some embodiments, the sealing features 1314 optionally may constrict the finger 1313, which may further increase capillary dilation. Larger diameter (i.e., dilated) capillaries and microvasculature increase local blood supply and reduce flow resistance after lancing, thereby providing an increased blood flow 1615 to the collection tube 107. As mentioned, inclusion of the finger seal 1328 may allow for improved blood flow 409 into the collection tube 107 while having minimal contact between blood (or other biological sample) and the material of the body 102.

In certain embodiments, a single compression/initiation of the vacuum may be utilized. However, in other embodiments such as where a sample is needed for multiple collection tubes 107, the capillary blood collection device 1601 may be further compressed to initiate successive vacuums, thereby providing the ability for a multi-draw capacity from the same draw site 1211 due to the increased blood flow.

Collecting a capillary blood sample utilizing the capillary blood collection devices described herein may provide various advantages compared to traditional fingerstick technologies. In some embodiments, the capillary blood collection devices may allow for an increased collection volume and a decreased collection time compared to traditional approaches. As a non-limiting example, a conventional fingerstick may obtain a collection volume of 100-600 μL over a collection time of 1-4 minutes, while the capillary blood collection devices described herein may obtain a collection volume of 1000-2050 μL over a collection time of 30-90 seconds. Moreover, while traditional techniques are not suitable for self-collection, the capillary blood collection devices described herein may be suitable for self-collection and assisted collection. The increased blood flow generated by the capillary blood collection devices described herein may further eliminate any need for preparation of the finger as conventionally required (e.g., rubbing/heating of the area, applying a warm pack to the area, etc.).

A collection of exemplary embodiments is provided below, including at least some explicitly enumerated as an “Illustration” providing additional description of a variety of example embodiments in accordance with the concepts described herein. These illustrations are not meant to be mutually exclusive, exhaustive, or restrictive; and the disclosure not limited to these example illustrations but rather encompasses all possible modifications and variations within the scope of the issued claims and their equivalents.

Illustration 1. A capillary blood collection device comprising an elongated body defining an internal chamber and comprising: a first end; a second end; and a port between the first end and the second end and providing access to the internal chamber between the first end and the second end, wherein the first end of the body defines an opening for receiving a human finger into the internal chamber, and wherein the second end of the body opposite from the first end is compressible.

Illustration 2. The capillary blood collection device of any preceding or subsequent illustration or combination of illustrations, wherein the second end is self-venting.

Illustration 3. The capillary blood collection device of any preceding or subsequent illustration or combination of illustrations, wherein the elongated body comprises an elastomeric material.

Illustration 4. The capillary blood collection device of any preceding or subsequent illustration or combination of illustrations, wherein the compressible material comprises silicone.

Illustration 5. The capillary blood collection device of any preceding or subsequent illustration or combination of illustrations, wherein the capillary blood collection device is monolithic.

Illustration 6. The capillary blood collection device of any preceding or subsequent illustration or combination of illustrations, wherein a center axis of the port is perpendicular to a center axis of the internal chamber.

Illustration 7. The capillary blood collection device of any preceding or subsequent illustration or combination of illustrations, wherein a diameter of the port is less than a diameter of the opening.

Illustration 8. The capillary blood collection device of any preceding or subsequent illustration or combination of illustrations, wherein the second end comprises a self-sealing air valve.

Illustration 9. The capillary blood collection device of any preceding or subsequent illustration or combination of illustrations, wherein the first end comprises a sealing feature for generating a seal with the human finger.

Illustration 10. The capillary blood collection device of any preceding or subsequent illustration or combination of illustrations, wherein the sealing feature comprises at least one rib on an inner surface of the internal chamber proximate to the first end,

Illustration 11. The capillary blood collection device of any preceding or subsequent illustration or combination of illustrations, wherein the body further comprises a finger seal within the internal chamber, wherein the finger seal is aligned with the port and extends from an inner surface of the internal chamber.

Illustration 12. A capillary blood collection device comprising a body, the body comprising an internal chamber for receiving a human finger and a port providing access to the internal chamber and configured to receive at least one of a lancet device or a collection tube, wherein the body is compressible and configured to generate a vacuum when compressed.

Illustration 13. The capillary blood collection device of any preceding or subsequent illustration or combination of illustrations, wherein a center axis of the port is perpendicular to a center axis of the internal chamber.

Illustration 14. The capillary blood collection device of any preceding or subsequent illustration or combination of illustrations, wherein the body is an elastomeric material.

Illustration 15. The capillary blood collection device of any preceding or subsequent illustration or combination of illustrations, wherein the body comprises a self-sealing air valve.

Illustration 16. The capillary blood collection device of any preceding or subsequent illustration or combination of illustrations, wherein the port is configured to receive both the lancet device and the collection tube.

Illustration 17. A method of collecting a blood sample, the method comprising: inserting a finger into an internal chamber of a capillary blood collection device; pressing a lancet into a port of the capillary blood collection device and lancing the finger within the internal chamber at a draw site; inserting a collection tube into the port; compressing the capillary blood collection device to initiate a vacuum within the internal chamber; and collecting the blood sample through the port and into the collection tube.

Illustration 18. The method of any preceding or subsequent illustration or combination of illustrations, further comprising collecting the blood sample for a collection time of 30-90 seconds.

Illustration 19. The method of any preceding or subsequent illustration or combination of illustrations, further comprising collecting the blood sample for a collection time of 1-5 minutes.

Illustration 20. A method of collecting at least 400 μL of a blood sample with a capillary blood collection device, the method comprising: inserting a finger into an internal chamber of a capillary blood collection device; pressing a lancet into a port of the capillary blood collection device and lancing the finger within the internal chamber at a draw site; inserting a collection tube into the port; compressing the capillary blood collection device to initiate a vacuum within the internal chamber; and collecting the blood sample through the port and into the collection tube for a collection time of 30-90 seconds.

Illustration 21. The method of any preceding or subsequent illustration or combination of illustrations, wherein the method comprises collecting 100-1000 μL during the collection time.

Illustration 22. The method of any preceding or subsequent illustration or combination of illustrations, wherein the method comprises collecting 1000-2050 μL during the collection time.

Illustration 23. A capillary blood collection device comprising a body comprising a first end for receiving a human finger into the capillary blood collection device, a compressible second end opposite from the first end, and a port between the first end and the second end, wherein the port is configured to receive a collecting accessory device.

Illustration 24. The capillary blood collection device of any preceding or subsequent illustration or combination of illustrations, wherein the compressible material comprises a hardness factor between 10 Shore A durometer and 70 Shore A durometer.

Illustration 25. A capillary blood collection device comprising a body defining an internal chamber and comprising: a first end; a second end; a port between the first end and the second end and providing access to the internal chamber between the first end and the second end, wherein the first end defines an opening into the internal chamber, wherein the second end of the body opposite from the first end is compressible. Optionally, the capillary blood collection device includes a flange at the first end is configured to engage skin of a user,

Illustration 26. The capillary blood collection device of any preceding or subsequent illustration or combination of illustrations, wherein the body is monolithic.

Illustration 27. The capillary blood collection device of any preceding or subsequent illustration or combination of illustrations, wherein the body comprises an elastomeric material.

Illustration 28. The capillary blood collection device of any preceding or subsequent illustration or combination of illustrations, wherein the compressible material comprises silicone.

Illustration 29. The capillary blood collection device of any preceding or subsequent illustration or combination of illustrations, wherein the compressible material comprises rubber, polyurethane, or thermoplastic elastomer.

Illustration 30. The capillary blood collection device of any preceding or subsequent illustration or combination of illustrations, further comprising a flange at the first end.

Illustration 31. The capillary blood collection device of any preceding or subsequent illustration or combination of illustrations, wherein the body comprises a material such that the first end is configured to conform to a skin surface of the user,

Illustration 32. The capillary blood collection device of any preceding or subsequent illustration or combination of illustrations, wherein the capillary blood collection device is adhesive-free,

Illustration 33. The capillary blood collection device of any preceding or subsequent illustration or combination of illustrations, wherein the first end is configured to adhere to the skin of the user via vacuum.

Illustration 34. The capillary blood collection device of any preceding or subsequent illustration or combination of illustrations, wherein the second end is a compressible bulb.

Illustration 35. The capillary blood collection device of any preceding or subsequent illustration or combination of illustrations, wherein the compressible bulb comprising a double-bulb and/or two or more bellows/lobes.

Illustration 36. The capillary blood collection device of any preceding or subsequent illustration or combination of illustrations, wherein the compressible bulb comprises a single hemisphere.

Illustration 37. The capillary blood collection device of any preceding or subsequent illustration or combination of illustrations, wherein the body further comprises a groove between the first end and the second end.

Illustration 38. The capillary blood collection device of any preceding or subsequent illustration or combination of illustrations, wherein a transverse dimension of the opening is greater than a transverse dimension of the port.

Illustration 39. The capillary blood collection device of any preceding or subsequent illustration or combination of illustrations, wherein the first end further comprises an integrated valve.

Illustration 40. The capillary blood collection device of any preceding or subsequent illustration or combination of illustrations, wherein the integrated valve comprises a slit in the body at the first end and/or a valve opening wherein a transverse dimension of the valve opening is less than a transverse dimension of the opening in the first end.

Illustration 41. The capillary blood collection device of any preceding or subsequent illustration or combination of illustrations, wherein the integrated valve comprises a valving feature with a flap and a channel extending from the body at the first end.

Illustration 42. The capillary blood collection device of any preceding or subsequent illustration or combination of illustrations, further comprising a gasket on the first end.

Illustration 43. The capillary blood collection device of any preceding or subsequent illustration or combination of illustrations, wherein a material of the gasket is different from a material of the body.

Illustration 44. The capillary blood collection device of any preceding or subsequent illustration or combination of illustrations, wherein the gasket defines a gasket opening, and wherein a transverse dimension of the gasket opening is less than a transverse dimension of the opening in the first end.

Illustration 45. The capillary blood collection device of any preceding or subsequent illustration or combination of illustrations, wherein the gasket further comprises a channel defined in the gasket,

Illustration 46. The capillary blood collection device of any preceding or subsequent illustration or combination of illustrations, wherein an axis of the port is substantially perpendicular to an axis of the opening.

Illustration 47. The capillary blood collection device of any preceding or subsequent illustration or combination of illustrations, wherein the opening is circular.

Illustration 48. The capillary blood collection device of any preceding or subsequent illustration or combination of illustrations, wherein the body comprises a material with a hardness of 10-80 Shore A durometer.

Illustration 49. The capillary blood collection device of any preceding or subsequent illustration or combination of illustrations, wherein the capillary blood collection device is configured to collect a volume of blood from 0.05 mL to 5.0 mL.

Illustration 50. The capillary blood collection device of any preceding or subsequent illustration or combination of illustrations wherein the capillary blood collection devices is configured to generate a pressure from −5 to −80 kPa.

Illustration 51. The capillary blood collection device of any preceding or subsequent illustration or combination of illustrations, wherein a transverse dimension of the opening is from 10 to 50 mm.

Illustration 52. The capillary blood collection device of any preceding or subsequent illustration or combination of illustrations, further comprising a flange at the first end, wherein a thickness of the flange is from 1.0 to 10.0 mm.

Illustration 53. The capillary blood collection device of any preceding or subsequent illustration or combination of illustrations, further comprising a flange at the first end and at least one removal tab extending outwards from the flange.

Illustration 54. The capillary blood collection device of any preceding or subsequent illustration or combination of illustrations, wherein a wall thickness of the body is from 1.0 to 8.0 mm.

Illustration 55. The capillary blood collection device of any preceding or subsequent illustration or combination of illustrations, wherein an inner surface of the body forming the inner chamber is tapered from the opening to the port.

Illustration 56. A capillary blood collection system comprising the capillary blood collection device of any preceding or subsequent illustration or combination of illustrations and a lancet, wherein the lancet is removable from and/or is not integrated with the capillary blood collection device.

Illustration 57. A capillary blood collection system comprising the capillary blood collection device of any preceding or subsequent illustration or combination of illustrations and a collection tube, wherein the collection tube is removable from the port,

Illustration 58. A method of collecting a blood sample, the method comprising: applying a capillary blood collection device to the skin of a user such that a lancet site is contained within an opening defined at a first end of the capillary blood collection device; inserting a collection tube into a port of the capillary blood collection device; compressing a second end of the capillary blood collection device to initiate a vacuum within the internal chamber and to cause the capillary blood collection device to adhere to the skin of the user; and collecting the blood sample through the port and into the collection tube.

Illustration 59. A method of collecting a blood sample, the method comprising: creating a wound on skin of a user at a lancet site; inserting a collection tube into a port of a capillary blood collection device; applying the capillary blood collection device to the skin of the user such that the lancet site is contained within an opening defined at a first end of the capillary blood collection device; compressing a second end of the capillary blood collection device, then releasing the compression to initiate a vacuum within the internal chamber and to cause the capillary blood collection device to adhere to the skin of the user; and collecting the blood sample through the port and into the collection tube.

Illustration 60. The method of any preceding or subsequent illustration or combination of illustrations, wherein the second end of the capillary blood collection device comprises a bulb and wherein compressing the second end of the capillary blood collection device is achieved by pressing the bulb toward the skin of the user while applying the capillary blood collection device to the skin of the user.

Illustration 61. The method of any preceding or subsequent illustration or combination of illustrations, wherein the method is performed by the user.

Illustration 62. The method of any preceding or subsequent illustration or combination of illustrations, wherein the method is performed by an assistant.

Illustration 63. The method of any preceding or subsequent illustration or combination of illustrations, further comprising collecting the blood sample for a collection time of up to about 4 minutes.

Illustration 64. The method of any preceding or subsequent illustration or combination of illustrations, wherein the collection tube is oriented downward during collection.

Illustration 65. The method of any preceding or subsequent illustration or combination of illustrations, wherein the lancet site is on the skin of the user on the upper arm, shoulder, lower back, forearm, thigh, palm of the hand, or back of the band,

Illustration 66. The method of any preceding or subsequent illustration or combination of illustrations, further comprising warming the skin of the user at a lancet site and wounding the skin of the user at the lancet site.

Illustration 67. The method of Illustration 58 wherein the skin of the user is warmed to a surface temperature between 40 C and 43 C.

Illustration 68. The method of any preceding or subsequent illustration or combination of illustrations, wherein a wound about 2 mm deep and 2 mm to 5 mm wide is created in the skin of the user at a lancing site.

Illustration 69. The method of any preceding or subsequent illustration or combination of illustrations, further comprising orienting the capillary blood collection device on the skin such that the port is oriented downwards,

Illustration 70. The method of any preceding or subsequent illustration or combination of illustrations, further comprising utilizing a plurality of lancets at a plurality of draw sites and/or a plurality of collection tubes for collecting the blood sample.

Illustration 71, A capillary blood collection device comprising a body comprising a first end for forming a seal against skin of a user and/or receiving a human finger into the capillary blood collection device, a compressible second end opposite from the first end, and a port between the first end and the second end, wherein the port is configured to receive a collecting accessory device.

As used herein, the meaning of “a,” “an,” and “the” includes singular and plural references unless the context clearly dictates otherwise.

The subject matter of embodiments of the present disclosure is described here with specificity to meet statutory requirements, but this description is not necessarily intended to limit the scope of the claims. The claimed subject matter may be embodied in other ways, may include different elements or steps, and may be used in conjunction with other existing or future technologies, This description should not be interpreted as implying any particular order or arrangement among or between various steps or elements except when the order of individual steps or arrangement of elements is explicitly described. Directional references such as “up,” “down,” “top,” “bottom,” “left,” “right,” “vertical,” “horizontal,” “lateral,” “longitudinal,” “front,” and “back,” among others, are intended to refer to the orientation as illustrated and described in the figure (or figures) to which the components and directions are referencing.

The terms “comprising,” “having,” “including,” and “containing” are to be construed as open-ended terms (i.e., meaning “including, but not limited to,”) unless otherwise noted. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention, and does not pose a limitation on the scope of the invention unless otherwise claimed. No language in the specification should be construed as indicating any non-claimed element as essential to the practice of the invention.

The above-described aspects are merely possible examples of implementations, merely set forth for a clear understanding of the principles of the present disclosure. Many variations and modifications can be made to the above-described embodiment(s) without departing substantially from the spirit and principles of the present disclosure. All such modifications and variations are intended to be included herein within the scope of the present disclosure, and all possible claims to individual aspects or combinations of elements or steps are intended to be supported by the present disclosure. Moreover, although specific terms are employed herein, as well as in the claims that follow, they are used only in a generic and descriptive sense, and not for the purposes of limiting the described embodiments, nor the claims that follow.

That which is claimed:

1. A capillary blood collection device comprising a body defining an internal chamber and comprising:
a first end;
a second end; and
a port between the first end and the second end and providing access to the internal chamber between the first end and the second end,
wherein the first end of the body defines an opening into the internal chamber, and
wherein the second end of the body opposite from the first end is compressible.

2. The capillary blood collection device of claim 1, wherein the body is self-venting.

3. The capillary blood collection device of claim 1, wherein the body comprises a compressible material.

4. The capillary blood collection device of claim 1, wherein the capillary blood collection device is monolithic.

5. The capillary blood collection device of claim 1, wherein a center axis of the port is perpendicular to a center axis of the internal chamber.

6. The capillary blood collection device of claim 1, wherein the capillary blood collection device is adhesive-free and wherein the body comprises a material such that the first end is configured to conform and adhere to a skin surface of a user via vacuum.

7. The capillary blood collection device of claim 1, wherein the second end is a compressible bulb.

8. The capillary blood collection device of claim 1, wherein a transverse dimension of the opening is greater than a transverse dimension of the port.

9. The capillary blood collection device of claim 1, wherein at least one of the first end or the second end further comprises an integrated valve.

10. The capillary blood collection device of claim 1, further comprising a gasket on the first end, wherein the gasket defines a gasket opening, and wherein a transverse dimension of the gasket opening is less than a transverse dimension of the opening in the first end.

11. The capillary blood collection device of claim 10, wherein the gasket further comprises a channel defined in the gasket.

12. The capillary blood collection device of claim 1, further comprising a sealing feature within the internal chamber proximate to the first end for generating a seal with a human finger positioned within the opening.

13. The capillary blood collection device of claim 1, wherein the body further comprises a finger seal within the internal chamber, wherein the finger seal is aligned with the port and extends from an inner surface of the internal chamber.

14. The capillary blood collection device of claim 1, further comprising a flange at the first end, wherein the flange is configured to engage skin of a user.

15. The capillary blood collection device of claim 14, further comprising at least one removal tab extending outwards from the flange.

16. The capillary blood collection device of claim 1, wherein an inner surface of the body forming the internal chamber is tapered from the opening to the port.

17. A capillary blood collection system comprising the capillary blood collection device of claim 1, a collection tube, and a lancet, wherein the collection tube is removable from the port, and wherein the lancet is removable from and/or is not integrated with the capillary blood collection device.

18. A capillary blood collection device comprising a body comprising:
a first end for forming a seal against skin of a user and/or receiving a human finger into the capillary blood collection device;
a compressible second end opposite from the first end; and
a port between the first end and the second end, wherein the port is configured to receive a collecting accessory device.

19. The capillary blood collection device of claim 18, wherein a center axis of the port is perpendicular to a center axis of an opening at the first end.

20. The capillary blood collection device of claim 18, wherein the body comprises a self-sealing air valve.
